(19) United States
(12) Patent Application Publication
(10) Pub. No.: US 2014/0174320 A1
(43) Pub. Date: Jun. 26, 2014

(54) IONIC LIQUID AND VISCOSE COMPOSITION

(71) Applicant: Taiwan Textile Research Institute, New Taipei City (TW)

(72) Inventors: Kuo-Liang Liu, Chiayi County (TW); Tung-Ung Chen, Taipei City (TW); Tai-Hong Cheng, New Taipei City (TW); Yu-Deng Lin, New Taipei City (TW)

(73) Assignee: Taiwan Textile Research Institute, New Taipei City (TW)

(21) Appl. No.: 14/097,268

(22) Filed: Dec. 5, 2013

Related U.S. Application Data
(60) Provisional application No. 61/733,427, filed on Dec. 5, 2012.

(30) Foreign Application Priority Data
Jan. 7, 2013 (TW) 102100467

(51) Int. Cl. C07D 233/60; C09D 7/00

(52) U.S. Cl. CPC C07D 233/60; C09D 7/001
USPC 106/166.3; 548/342.1

(57) ABSTRACT

An ionic liquid, comprising a compound of Formula 1:

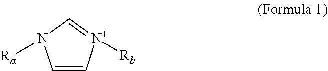

wherein Rₐ is a hydrocarbon chain having 2 to 10 carbon atoms and a terminal hydroxyl group and the hydrocarbon chain comprising —O— therein, —CO— therein or comprising both —O— and —CO— therein, R_{b} is —CₓH_{y}SO₃H, —CₓH_{y}PO₃H, —CₓH_{y}COOH, —CₓH_{y}SO₃⁻, —CₓH_{y}PO₃⁻ or —CₓH_{y}COO⁻, and x ranges from 1 to 6 and y ranges from 2 to 12.

### CROSS-REFERENCE TO RELATED APPLICATION

This application claims the priority benefits of U.S. provisional application Ser. No. 61/733,427, filed on Dec. 5, 2012, and Taiwan application serial No. 102100467, filed on Jan. 7, 2013. The entirety of each of the above-mentioned patent applications is hereby incorporated by reference herein and made a part of this specification.

### BACKGROUND OF THE INVENTION

1. Field of the Invention

The present invention relates to an ionic liquid and a viscose composition and, more particularly, to an ionic liquid capable of dissolving polymeric materials and the viscose composition thereof.

2. Description of Related Art

The resources of the earth and the environment have been excessively exploited and destructed by human for a long time, which results in the gradual depletion of natural resources. In recent years, scientists are keen to develop environmental friendly alternative energy and waste recycling technology, in order to mitigate the gradual depletion of natural resources. Regenerated cellulose manufactured through dissolving the existing polymeric material may replace natural cellulose so as to reduce the consumption of natural cellulose. Regenerated cellulose is widely used in various industries, including home furnishing textile products, optical thin films, flexible electronic displays, cosmetics, pharmaceutical products and food additives.

Taking the manufacturing process of regenerated cellulose as the example, it is necessary to dissolve the existing cellulose material(s) to form a cellulose solution for the subsequent manufacturing processes. Currently, solvents mainly used to dissolve the cellulose material(s) are carbon disulfide and N-methylmorpholine-N-oxide (NMMO). It is required that N-methylmorpholine-N-oxide is heated to a high temperature (about 110° C.) to well dissolve the cellulose material(s). However, the manufacturing process of high temperature results in the degradation of the cellulose material(s). Besides, as N-methylmorpholine-N-oxide is usually employed along with the stabilizer, it is not easy to recycle the solvent. In summary, it is desirable to develop a solvent having good solubility and capable of being recycled and reused in this field.

### SUMMARY OF THE INVENTION

The present invention provides an ionic liquid with good solubility for polymeric material. The present invention provides a viscose composition, comprising the aforementioned ionic liquid.

The present invention provides an ionic liquid, comprising a compound of Formula 1:

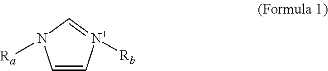

wherein Rₐ is a hydroxyl group comprising —O— therein and having 2 to 10 carbon atoms, a hydroxyl group comprising —CO— therein and having 2 to 10 carbon atom, or a hydroxyl group comprising both —O— and —CO— therein and having 2 to 10 carbon atoms, R_{b} is —CₓH_{y}SO₃H, —CₓH_{y}PO₃H, —CₓH_{y}COOH, —CₓH_{y}SO₃⁻, —CₓH_{y}PO₃⁻ or —CₓH_{y}COO⁻, and x ranges from 1 to 6 and y ranges from 2 to 12.

The present invention provides a viscose composition, comprising at least one ionic liquid and at least one polymeric material, wherein the polymeric material is dissolved in the ionic liquid. The ionic liquid comprises a compound of Formula 1:

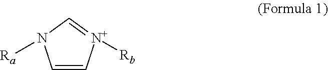

wherein Rₐ is a hydroxyl group comprising —O— therein and having 2 to 10 carbon atoms, a hydroxyl group comprising —CO— therein and having 2 to 10 carbon atom, or a hydroxyl group comprising both —O— and —CO— therein and having 2 to 10 carbon atoms. R_{b} is —CₓH_{y}SO₃H, —CₓH_{y}PO₃H, —CₓH_{y}COOH, —CₓH_{y}SO₃⁻, —CₓH_{y}PO₃⁻ or —CₓH_{y}COO⁻, and x ranges from 1 to 6 and y ranges from 2 to 12.

The present invention provides an ionic liquid, comprising a nitrogen-containing compound having a five-membered ring, a nitrogen-containing six-membered ring or a nitrogen-substituted naphthalene structure, wherein the nitrogen-containing compound having the five-membered ring comprises one selected from Formula (a) to Formula (h), the nitrogen-containing compound having the six-membered ring comprises one selected from Formula (i) to Formula (m), and the nitrogen-containing compound having the nitrogen-substituted naphthalene structure comprises one selected from Formula (n) to Formula (o).

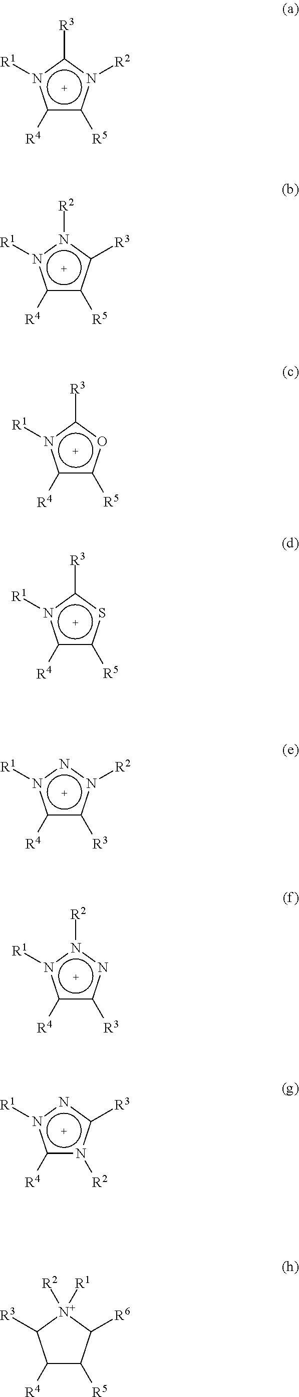

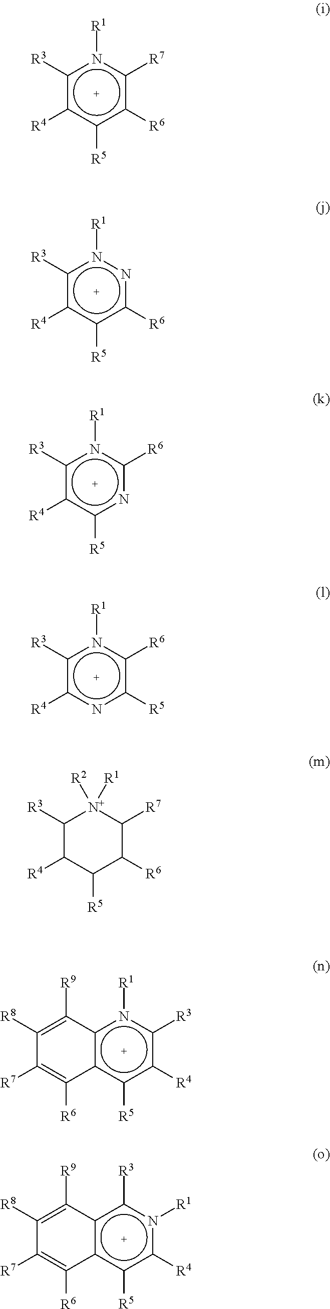

wherein R¹ and R² are individually

—CₓH_{y}SO₃⁻, —CH_{y}PO₃H⁻, —(CₓH_{y})(PO₃H₂)PO₃H⁻, —CₓH_{y}COO⁻, —CₓH_{y}SO₃H, —CₓH_{y}PO₃H₂, —(CₓH_{y})(PO₃H₂)PO₃H₂ or —CₓH_{y}COOH, and x ranges from 1 to 6 and y ranges from 2 to 12;

R¹ to R⁹ are each individually

Hydrido;

—(Z¹)—O—(Z²)—OH, wherein Z¹ and Z² are individually alkylene with 1 to 6 carbon atoms, or substituted alkylene with 1 to 6 carbon atoms;

—(Z³)—O—Z⁴, wherein Z³ is the substituted or unsubstituted alkylene with 1 to 6 carbon atoms, and Z⁴ is the substituted or unsubstituted alkylene with 1 to 6 carbon atoms; or

a first group or a second group, wherein the first group and the second group are individually alkyl with 1 to 6 carbon atoms, alkenyl with 1 to 6 carbon atoms, alkene with 1 to 6 carbon atoms, alkylene with 1 to 6 carbon atoms, aryl with 1 to 6 carbon atoms, cycloalkyl with 1 to 6 carbon atoms or cyclic group with 1 to 6 carbon atoms, and the second group is substituted by one of the following substituted groups consisting of alkoxy, alkenyl, alkynyl, aryl, heteroaryl, aldehyde, amino, ester, ether, halide, hydroxyl, ketone, nitro, silyl, sulfo-oxo, sulfonyl, sulfone, sulfoxide, sulfate and thiol.

The aforementioned ionic liquid of the present invention comprises the positively charged nitrogen-containing compound having the five-membered ring with the electron resonance structure. In addition, the ionic liquid of the present invention comprises highly polar side chain of hydroxyl group(s). Therefore, the ionic liquid of the present invention can effectively form bonding with the dissolved polymeric materials, so the polymeric material solubility is good.

The concepts and advantages of the present invention will become more apparent in view of the following embodiments, with drawings and accompanying detailed information.

### DESCRIPTION OF THE EMBODIMENTS

### Ionic Liquid

The ionic liquid of the present embodiment comprises a nitrogen-containing compound having a five-membered ring, a six-membered ring or a nitrogen-substituted naphthalene structure, wherein

the nitrogen-containing compound having the five-membered ring comprises one selected from Formula (a) to Formula (h),

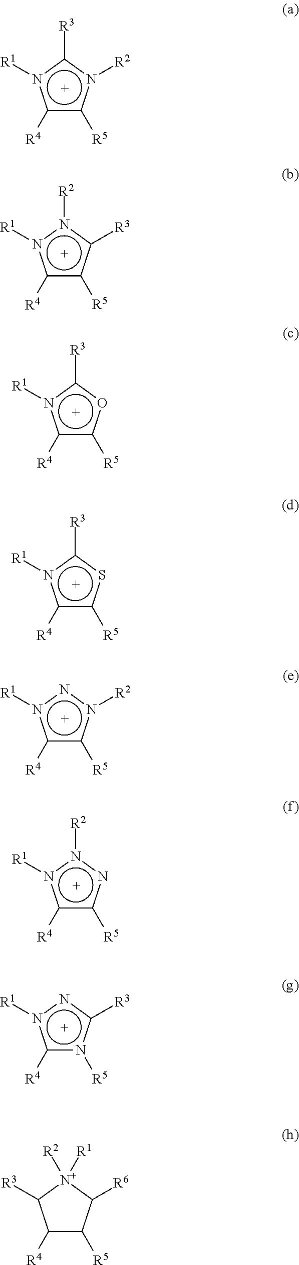

the nitrogen-containing compound having the six-membered ring comprises one selected from Formula (i) to Formula (m),

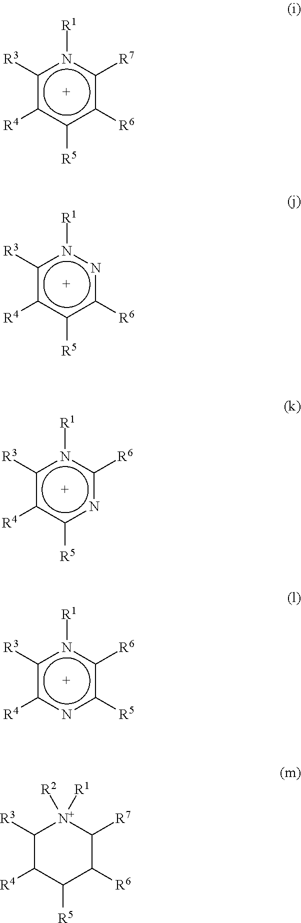

the nitrogen-containing compound having the nitrogen-substituted naphthalene structure comprises one selected from Formula (n) to Formula (o),

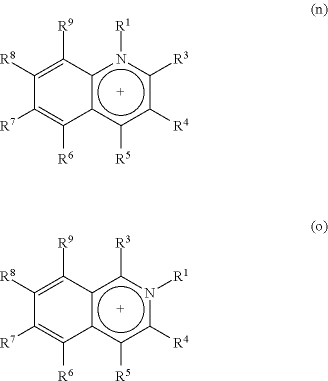

wherein, R¹ and R² are individually

—CₓH_{y}SO₃⁻, —CₓH_{y}PO₃H—, —(CₓH_{y})(PO₃H₂)PO₃H⁻, —CₓH_{y}COO⁻, —CₓH_{y}SO₃H, —CₓH_{y}PO₃H₂, —(CₓH_{y})(PO₃H₂)PO₃H₂ or —CₓH_{y}COOH, and x ranges from 1 to 6 and y ranges from 2 to 12;

R¹ to R⁹ are each individually

Hydrido;

—(Z¹)—O—(Z²)—OH, wherein Z¹ and Z² are individually alkylene with 1 to 6 carbon atoms, or substituted alkylene with 1 to 6 carbon atoms;

—(Z³)—O—Z⁴, wherein Z³ is the substituted or unsubstituted alkylene with 1 to 6 carbon atoms, and Z⁴ is the substituted or unsubstituted alkylene with 1 to 6 carbon atoms; or

a first group or a second group, wherein the first group and the second group are individually alkyl with 1 to 6 carbon atoms, alkenyl with 1 to 6 carbon atoms, alkene with 1 to 6 carbon atoms, alkylene with 1 to 6 carbon atoms, aryl with 1 to 6 carbon atoms, cycloalkyl with 1 to 6 carbon atoms or cyclic group with 1 to 6 carbon atoms, and the second group is substituted by one of the following substituted groups consisting of alkoxy, alkenyl, alkynyl, aryl, heteroaryl, aldehyde, amino, ester, ether, halide, hydroxyl, ketone, nitro, silyl, sulfo-oxo, sulfonyl, sulfone, sulfoxide, sulfate and thiol.

In one embodiment, the ionic liquid comprises the compound of Formula 8:

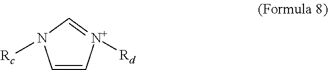

wherein R_{c} is —(Z¹)—O—(Z²)—OH, and Z¹ and Z² are individually alkylene with 1 to 6 carbon atoms, or substituted alkylene with 1 to 6 carbon atoms; R_{d} is —CₓH_{y}SO₃H, —CₓH_{y}PO₃H, —CₓH_{y}COOH, —CₓH_{y}SO₃⁻ or —CₓH_{y}COO⁻, and x ranges from 1 to 6 and y ranges from 2 to 12.

In one embodiment, the ionic liquid comprises the compound of Formula 9:

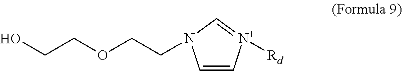

wherein R_{d} is —CₓH_{y}SO₃⁻ or —CₓH_{y}COO⁻, and x ranges from 1 to 6 and y ranges from 2 to 12.

In one embodiment, the ionic liquid comprises the compound of Formula 10:

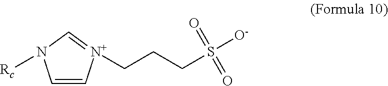

In one embodiment, the ionic liquid comprises the compound of Formula 11:

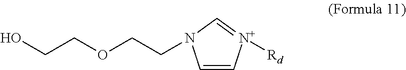

wherein R_{d} is —CₓH_{y}SO₃H, —CₓH_{y}PO₃H or —CₓH_{y}COOH, and x ranges from 1 to 6 and y ranges from 2 to 12.

In one embodiment, the ionic liquid further comprises an anion, and the anion comprises halogen ion, perchlorate, pseudohalogen ion, thiocyanate, cyanate, ClO₄⁻, BF₄⁻, PF₆⁻, AsF₆⁻, SbF₆⁻, NO₃⁻, NO₂⁻, SO₄²⁻, HSO₄⁻, PO₄³⁻, HPO₄²⁻, H₂PO₄⁻, CF₃CO₂⁻, CO₃²⁻, citrate, carboxylate with 1 to 6 carbon atoms and —CₓH_{y}COO, and x ranges from 1 to 6 and y ranges from 2 to 12.

In one embodiment, the compound of the ionic liquid is represented by Formula 1:

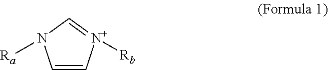

wherein Rₐ is a hydroxyl group comprising —O— therein and having 2 to 10 carbon atoms, a hydroxyl group comprising —CO— therein and having 2 to 10 carbon atom, or a hydroxyl group comprising both —O— and —CO— therein and having 2 to 10 carbon atoms, R_{b} is —CₓH_{y}SO₃H, —CₓH_{y}PO₃H, —CₓH_{y}COOH, —CₓH_{y}SO₃⁻, —CₓH_{y}PO₃⁻ or —CₓH_{y}COO⁻, and x ranges from 1 to 6 and y ranges from 2 to 12.

Generally, certain polymeric materials includes a plurality of groups of hydrogen bond donors or a plurality of groups of hydrogen bond acceptors, so that partial charges or electric dipole moment within molecules is formed, leading to certain extent of inner molecule polarity for the polymeric material. The aforementioned polymeric material comprises cellulose, lignocelluloses, hemicelluloses, lignin, polysaccharides, chitin, chitosan or a mixture thereof. Or, the aforementioned polymeric material comprises polyethylene, polypropylene, polyurethane, polyamide, polycaprolactam, poly-p-phenylene terephthamide, polyimide, polyethylene terephthalate or a mixture thereof.

In the present embodiment, Rₐ of the compound of the ionic liquid is a highly polar side chain group. Rₐ is the hydroxyl group comprising —O— therein and having 2 to 10 carbon atoms, a hydroxyl group comprising —CO— therein and having 2 to 10 carbon atom, or a hydroxyl group comprising both —O— and —CO— therein and having 2 to 10 carbon atoms, where the hydroxyl group enhances the polarity of the ionic liquid, and the chain structure comprising —O—, —CO— therein or comprising both —O— and —CO— therein can function as hydrogen bond donors or hydrogen bond acceptors. Hence, it is helpful to form bonding between the ionic liquid molecules and the dissolved polymeric materials, so that the dissolved polymeric material can evenly disperse and dissolve in the ionic liquid. Besides, Rₐ is a hydroxyl group having 2 to 10 carbon atoms. More particularly, exclusive of —O— or —CO— or both included therein, Ra, is the hydroxyl group having 2 to 10 carbon atoms. It is noted that, when the carbon chain of the hydrocarbon chain having the hydroxyl group is shorter, the solubility of the polymeric material within the ionic liquid for is better.

The ionic liquid of the present embodiment comprises a nitrogen-containing compound having a five-membered ring having the positive charge(s) and electron resonance structure. Hence, the polarity of the ionic liquid is further enhanced, and the solubility of the polymeric material in the ionic liquid is effectively improved.

R_{b} of the compound of the ionic liquid of the present embodiment is a side chain group comprising an alkanoate, wherein the hydrogen and oxygen atoms of alkanoate function as the hydrogen bond acceptors and hydrogen bond donors. Hence, the solubility of the polymeric material in the ionic liquid is further enhanced.

In one embodiment of the present invention, the compound of the ionic liquid is of Formula 2:

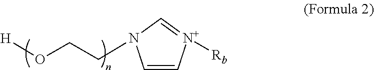

wherein n ranges from 1 to 5, R_{b} is —CₓH_{y}SO₃⁻, —CₓH_{y}PO₃⁻ or —CₓH_{y}COO⁻, x ranges from 1 to 6, and y ranges from 2 to 12. More particularly, R_{b} is the oxygen-containing alkanoate group with negative charge(s). Therefore, the ionic liquid of the present embodiment simultaneously comprises a nitrogen-containing compound having a five-membered ring with positive charge(s) and R_{b} being the side chain group with negative charge(s), resulting in the highly polar ionic liquid, and the solubility of the polymeric material in the ionic liquid is also enhanced.

In one embodiment, Rₐ of the compound of the ionic liquid is a hydroxyl group comprising —O— therein, and R₂ is —C₃H₆SO₃⁻, for the compound of Formula 3:

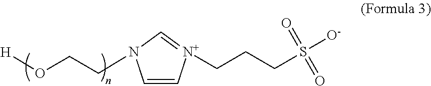

wherein n ranges from 1 to 5.

In another embodiment, Rₐ of the compound of the ionic liquid is a hydroxyl group comprising —O— therein, n is 2, and R_{b} is —C₃H₆SO₃⁻, for the compound of

Formula 4:

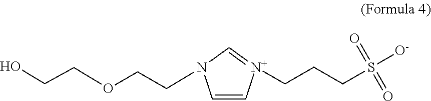 [SMILES: O=S(=O)([O-])CCC[n+]1ccn(CCOCCO)c1]

In the structure of Formula 4, Rₐ is described as a hydroxyl group having 4 carbon atoms and —O— therein as the example, but the present invention is not limited thereby. In the other embodiments, the compound of the ionic liquid may be also represented by Formula 5:

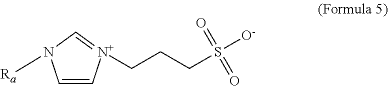

wherein Rₐ is a hydroxyl group comprising —O— therein and having 2 to 10 carbon atoms, a hydroxyl group comprising —CO— therein and having 2 to 10 carbon atom, or a hydroxyl group comprising both —O— and —CO— therein and having 2 to 10 carbon atoms.

For the compounds from Formula 2 to Formula 5, the ionic liquid comprises a nitrogen-containing compound having a five-membered ring with positive charge(s) and R_{b} being the side chain group with negative charge(s). However, the present invention is not limited thereby. In the other embodiments, the ionic liquid also comprises a nitrogen-containing compound having a five-membered ring with positive charge(s) and R_{b} being the side chain group with no charge(s).

For example, the compound of the ionic liquid is of Formula 6:

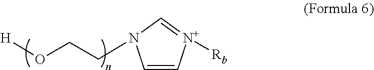

wherein n ranges from 1 to 5, R_{b} is —CₓH_{y}SO₃H, —CₓH_{y}PO₃H or —CₓH_{y}COOH, x ranges from 1 to 6, and y ranges from 2 to 12.

In the present embodiment, the ionic liquid further comprises an anion, wherein the anion is Cl⁻, Br⁻, I⁻, ClO₄⁻, BF₄⁻, PF₆⁻, AsF₆⁻, SbF₆⁻, NO₃⁻, NO₂⁻, SO₄²⁻, HSO₄⁻, PO₄³⁻, HPO₄²⁻, H₂PO₄⁻, CF₃CO₂⁻, CO₃²⁻, citrate, or carboxylate with 1 to 6 carbon atoms.

As the aforementioned, in one embodiment, the compound of the ionic liquid is of Formula 7:

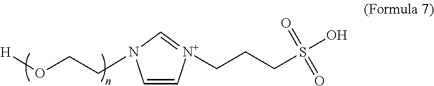

wherein n ranges from 1 to 5.

### <Viscose Composition>

The aforementioned ionic liquid is used as solvent. For example, the ionic liquid is used to dissolve the polymeric material and form a viscose composition. The dissolving temperature of the polymeric material ranges from 0° C. to 120° C., and the preferred temperature ranges from 35° C. to 90° C. Hence, since the dissolving temperature is low, it is not easy for the polymeric material to degrade during the dissolving process. The content of the polymeric material in the viscose composition ranges from 0.01 wt % (weight percentage) to 35 wt %, and the preferred content ranges from 5 wt % to 25 wt %.

In the present embodiment, the viscose composition comprises an ionic liquid and a polymeric material. In the other embodiment, an ionic liquid is used to dissolve more than two types of polymeric materials, in order to form a viscose composition for a complex material. Or, more than two kinds of ionic liquids are used as the mixture solvent for dissolving one or more than two kinds of polymeric materials. In other words, the types or kinds of the ionic liquid and the polymeric materials included in the viscose composition are not limited in the present invention.

The aforementioned polymeric material comprises cellulose, lignocelluloses, hemicelluloses, lignin, polysaccharide, chitin, chitosan or a mixture thereof. Alternatively, polymeric material comprises polyethylene, polypropylene, polyurethane, polyamide, polycaprolactam, poly-p-phenylene terephthamide, polyimide, polyethylene terephthalate or a mixture thereof.

Since the ionic liquid is water soluble, and it can be recycled and reused, having no volatility but of high thermal stability and chemical stability, the viscose composition produced by dissolving the polymeric material with the ionic liquid as described in the present embodiment has good workability, which is beneficial to the production of cellulose and thin films.

### <The Application of Viscose Composition>

Spinning Viscose

The viscose composition formed by dissolving the polymeric material in the ionic liquid of the present invention can be used as the spinning viscose for the wet type spinning. The polymeric material comprises cellulose, lignocelluloses, hemicelluloses, lignin, polysaccharide, chitin, chitosan or a mixture thereof. Alternatively, the polymeric material comprises polyethylene, polypropylene, polyurethane, polyamide, polycaprolactam, poly-p-phenylene terephthamide, polyimide, polyethylene terephthalate or a mixture thereof.

More particularly, taking cellulose material as an example, the pretreatment process may be performed firstly, and cleaning the surface impurity on the cellulose material and bleaching treatment are performed. Afterwards, the refinement process of the cellulose material is performed, so that the cellulose material is purified and then ground and crushed. Afterwards, the refined cellulose material is dissolved in the ionic liquid of the present invention to form the cellulose viscose. After adjusting the properties such as the concentration and viscosity of the cellulose viscose, the cellulose viscose can be used as the spinning viscose for the wet type spinning. The cellulose viscose is shaped into fibers through the spinning process. Afterwards, the stretching strength of the fibers is enhanced and the residues are washed off through the subsequent manufacturing process including the stretching process and water-washing process. Afterwards, performing the drying process to form the regenerated cellulose fibers.

After completing the spinning process, the residual shaping solution may contain small amounts of the polymeric material and large amounts of the ionic liquid. At this time, the ionic liquid can be further recycled and reused. The ionic liquid of the present invention has no volatility but high thermal stability, which facilitates the recycling of the ionic liquid for reuses.

Thin Film

The viscose composition can be applied for the production of thin films. The polymeric material of the viscose composition comprises cellulose, lignocelluloses, hemicelluloses, lignin, polysaccharide, chitin, chitosan or a mixture thereof. Alternatively, the polymeric material comprises polyethylene, polypropylene, polyurethane, polyamide, polycaprolactam, poly-p-phenylene terephthamide, polyimide, polyethylene terephthalate or a mixture thereof.

More particularly, the viscose composition is coated onto the glass substrate and then placed in water for a few minutes. The thin film is formed through the replacement of the ionic liquid by water. The aforementioned thin film produced by the polymeric material can be used as a filtration material, an insulation material, a transparent material or a filling material.

### Experiments

The preparation of the ionic liquid:

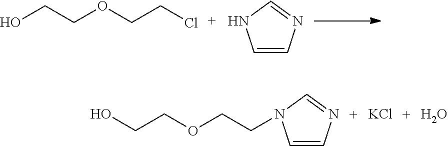

First, add imidazole and KOH into acetonitrile, then add 2-(2-chloroethoxy)ethanol, and then perform heating and reflux for 12 hours.

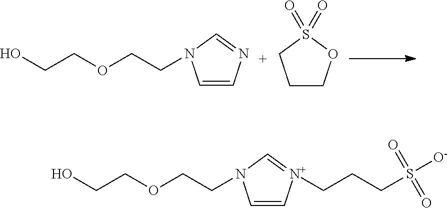

Afterwards, dissolve 2-[2-(1H-imidazol-1-yl)ethoxy]ethanol into toluene, then slowly add propanesultone, and then react 2 hours at room temperature. After the reaction is completed, the ionic liquid: 3-{1-[2-(2-hydroxyethoxy)ethyl]-1H-imidazol-3-ium-3-yl}propane-1-sulfonate is formed.

The identification of the ionic liquid:

Nuclear magnetic resonance spectroscopy (NMR) of the ionic liquid: 3-{1-[2-(2-hydroxyethoxy)ethyl]-1H-imidazol-3-ium-3-yl}propane-1-sulfonate is analyzed as the followings:

¹H NMR (200 MHz, D₂O): δ 2.39 (m, 2H), 2.42 (t, 2H), 3.45 (t, 2H), 3.51 (t, 2H), 3.70 (t, 2H), 4.26 (t, 2H), 4.30 (t, 2H), 7.64 (s, 1H), 7.76 (s, 1H), 9.10 (s, 1H). It is confirmed that the preparation of the ionic liquid is completed through the analysis of NMR.

The preparation of the viscose composition:

The cotton cellulose is added into the aforementioned prepared ionic liquid, then the mixture is heated for a few hours under the temperature of 70° C. to 85° C., and then a light yellow clear cellulose viscose composition is formed. At 85° C., the dissolved amount of cellulose in the cellulose viscose composition is about 25 wt %.

The preparation of cellulose thin films:

After coating 15 wt % cellulose viscose composition onto the glass substrate, it is placed in water for 2 to 10 minutes. The transparent cellulose thin film is formed through the replacement of the ionic liquid by water at room temperature.

In summary, because the ionic liquid of the present invention comprises the nitrogen-containing compound having the five-membered ring with positive charge(s) and with highly polar side chain group(s), strong bonding is effectively formed between the ionic liquid and the polymeric materials, and the solubility is good. In addition, the viscose composition of the present invention is formed by dissolving the polymeric material within the aforementioned ionic liquid. The ionic liquid of the present invention has good solubility, thermal stability and chemical stability, and it is unnecessary to add the stabilizer additionally when using the ionic liquid as the solvent, which is beneficial for recycling and reuse. Besides, the dissolving temperature of the viscose composition of the present invention is low, so it is unlikely to cause degradation of the polymeric material.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

1. An ionic liquid, comprising a compound of Formula 1: 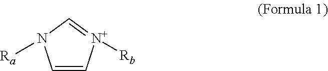
wherein Rₐ is a hydrocarbon chain having 2 to 10 carbon atoms and a terminal hydroxyl group and the hydrocarbon chain comprising —O— therein, —CO— therein or comprising both —O— and —CO— therein, R_{b} is —CₓH_{y}SO₃H, —CₓH_{y}PO₃H, —CₓH_{y}COOH, —CₓH_{y}SO₃⁻, —CₓH_{y}PO₃⁻ or —CₓH_{y}COO⁻, and x ranges from 1 to 6 and y ranges from 2 to 12.

2. The ionic liquid according to claim 1, wherein the compound of the ionic liquid is of Formula 2: 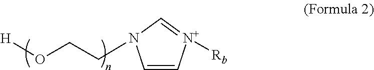
wherein n ranges from 1 to 5, R_{b} is —CH_{y}SO₃⁻, —CₓH_{y}PO₃⁻ or —CₓH_{y}COO⁻, x ranges from 1 to 6, and y ranges from 2 to 12.

3. The ionic liquid according to claim 2, wherein the compound of the ionic liquid is of Formula 3: 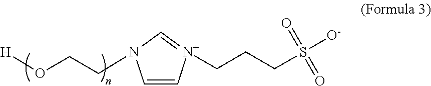
wherein n ranges from 1 to 5.

4. The ionic liquid according to claim 3, wherein the compound of the ionic liquid is of Formula 4:

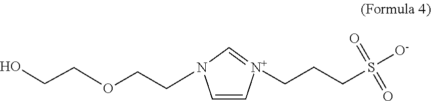 [SMILES: O=S(=O)([O-])CCC[n+]1ccn(CCOCCO)c1]

5. The ionic liquid according to claim 2, wherein the compound of the ionic liquid is of Formula 5: 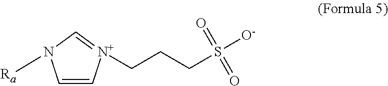
wherein Rₐ is a hydrocarbon chain having 2 to 10 carbon atoms and a terminal hydroxyl group and the hydrocarbon chain comprising —O— therein, —CO— therein or comprising both —O— and —CO— therein.

6. The ionic liquid according to claim 1, wherein the compound of the ionic liquid is of Formula 6: 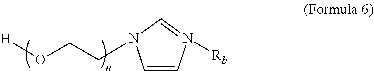
wherein n ranges from 1 to 5, R_{b} is —CₓH_{y}SO₃H, —CₓH_{y}PO₃H or —CₓH_{y}COOH, x ranges from 1 to 6, and y ranges from 2 to 12.

7. The ionic liquid according to claim 6, wherein the ionic liquid further comprises an anion, and the anion comprises Cl⁻, Br⁻, I⁻, ClO₄⁻, BF₄⁻, PF₆⁻, AsF₆⁻, SbF₆⁻, NO₃⁻, NO₂⁻, SO₄²⁻, HSO₄⁻, PO₄³⁻, HPO₄²⁻, H₂PO₄⁻, CF₃CO₂⁻, CO₃²⁻, citrate, or carboxylate with 1 to 6 carbon atoms.

8. The ionic liquid according to claim 6, wherein the compound of the ionic liquid is of Formula 7: 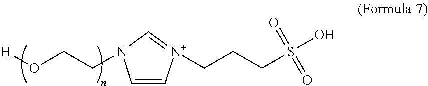
wherein n ranges from 1 to 5.

9. A viscose composition, comprising:
at least one polymeric material; and
at least one ionic liquid, and the polymeric material is dissolved in the ionic liquid, wherein the ionic liquid comprises a compound of Formula 1: 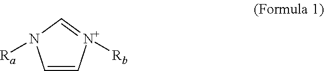
wherein Rₐ is a hydrocarbon chain having 2 to 10 carbon atoms and a terminal hydroxyl group and the hydrocarbon chain comprising —O— therein, —CO— therein or comprising both —O— and —CO— therein, R_{b} is —CₓH_{y}SO₃H, —CₓH_{y}PO₃H, —CₓH_{y}COOH, —CₓH_{y}SO₃⁻, —CₓH_{y}PO₃⁻ or —CₓH_{y}COO⁻, and x ranges from 1 to 6 and y ranges from 2 to 12.

10. The viscose composition according to claim 9, wherein a content of polymeric material in the viscose composition ranges from 0.01 wt % to 35 wt %.

11. The viscose composition according to claim 9, wherein a content of polymeric material in the viscose composition ranges from 5 wt % to 25 wt %.

12. The viscose composition according to claim 9, wherein the compound of the ionic liquid is of Formula 2: 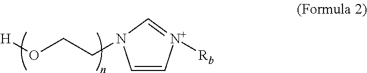
wherein n ranges from 1 to 5, R_{b} is —CₓH_{y}SO₃⁻, —CH_{y}PO₃⁻ or —CₓH_{y}COO⁻, x ranges from 1 to 6, and y ranges from 2 to 12.

13. The viscose composition according to claim 12, wherein the compound of the ionic liquid is of Formula 3: 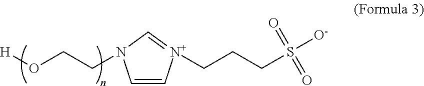
wherein n ranges from 1 to 5.

14. The viscose composition according to claim 13, wherein the compound of the ionic liquid is of Formula 4:

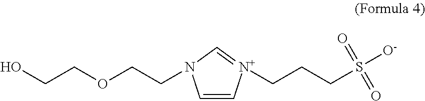 [SMILES: O=S(=O)([O-])CCC[n+]1ccn(CCOCCO)c1]

15. The viscose composition according to claim 12, wherein the compound of the ionic liquid is of Formula 5: 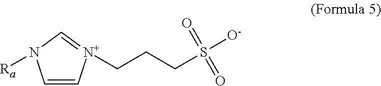
wherein Rₐ is a hydrocarbon chain having 2 to 10 carbon atoms and a terminal hydroxyl group and the hydrocarbon chain comprising —O— therein, —CO— therein or comprising both —O— and —CO— therein.

16. The viscose composition according to claim 9, wherein the compound of the ionic liquid is of Formula 6: 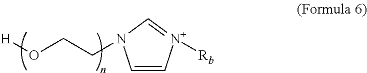
wherein n ranges from 1 to 5, R_{b} is —CₓH_{y}SO₃H, —CₓH_{y}PO₃H or —CₓH_{y}COOH, x ranges from 1 to 6, and y ranges from 2 to 12.

17. The viscose composition according to claim 16, wherein the ionic liquid further comprises an anion, and the anion comprises Cl⁻, Br⁻, I⁻, ClO₄⁻, BF₄⁻, PF₆⁻, AsF₆⁻, SbF₆⁻, NO₃⁻, NO₂⁻, SO₄²⁻, HSO₄⁻, PO₄³⁻, HPO₄²⁻, H₂PO₄⁻, CF₃CO₂⁻, CO₃²⁻, citrate, or carboxylate with 1 to 6 carbon atoms.

18. The viscose composition according to claim 16, wherein the compound of the ionic liquid is of Formula 7: 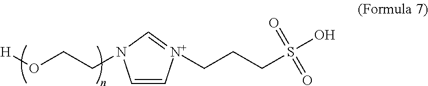
wherein n ranges from 1 to 5.

19. The viscose composition according to claim 9, wherein the polymeric material comprises cellulose, lignocelluloses, hemicelluloses, lignin, polysaccharide, chitin, chitosan or a mixture thereof.

20. The viscose composition according to claim 9, wherein the polymeric material comprises polyethylene, polypropylene, polyurethane, polyamide, polycaprolactam, poly-p-phenylene terephthamide, polyimide, polyethylene terephthalate or the a mixture thereof the above.

21. An ionic liquid, comprising a nitrogen-containing compound having a five-membered ring, a six-membered ring or a nitrogen-substituted naphthalene structure, wherein
the nitrogen-containing compound having the five-membered ring comprises one selected from Formula (a) to Formula (h), 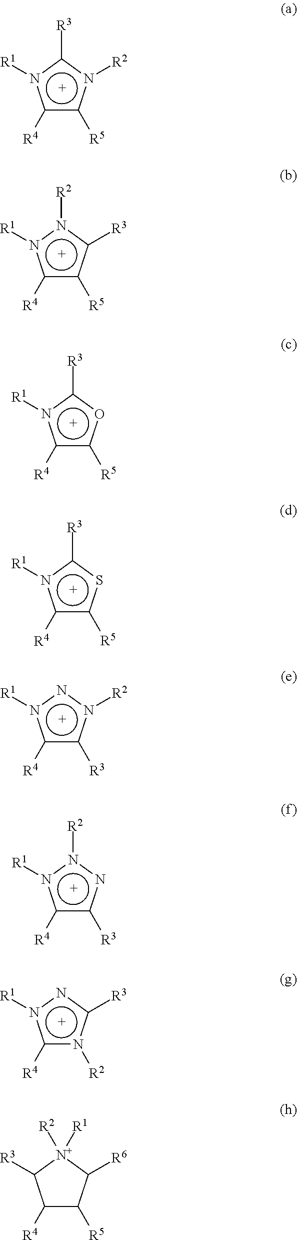
the nitrogen-containing compound having the six-membered ring comprises one selected from Formula (i) to Formula (m), 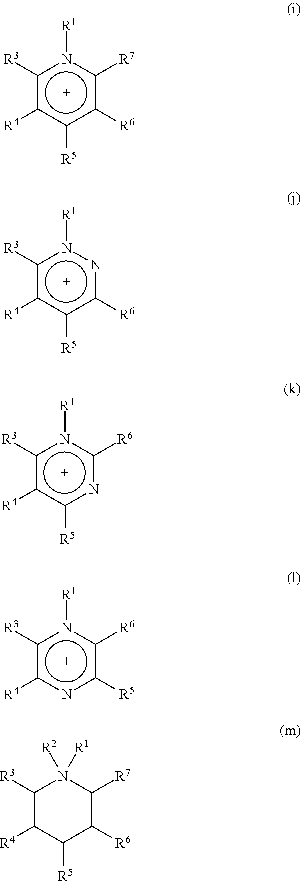
the nitrogen-containing compound having the nitrogen-substituted naphthalene structure comprises one selected from Formula (n) to Formula (o), 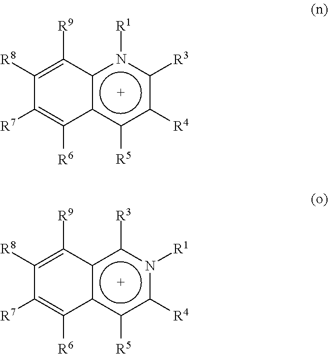
wherein, R¹ and R² are individually
—CₓH_{y}SO₃⁻, —CₓH_{y}PO₃H⁻, —(CₓH_{y})(PO₃H₂)PO₃H⁻, —CₓH_{y}COO⁻, —CₓH_{y}SO₃H, —CₓH_{y}PO₃H₂, —(CₓH_{y})(PO₃H₂)PO₃H₂ or —CₓH_{y}COOH, x ranges from 1 to 6, and y ranges from 2 to 12;
R¹ to R⁹ are each individually
Hydrido;
—(Z¹)—O—(Z²)—OH, wherein Z¹ and Z² are individually alkylene with 1 to 6 carbon atoms, or substituted alkylene with 1 to 6 carbon atoms;
—(Z³)—O—Z⁴, wherein Z³ is the substituted or unsubstituted alkylene with 1 to 6 carbon atoms, and Z⁴ is the substituted or unsubstituted alkylene with 1 to 6 carbon atoms; or
a first group or a second group, wherein the first group and the second group are individually alkyl with 1 to 6 carbon atoms, alkenyl with 1 to 6 carbon atoms, alkene with 1 to 6 carbon atoms, alkylene with 1 to 6 carbon atoms, aryl with 1 to 6 carbon atoms, cycloalkyl with 1 to 6 carbon atoms or cyclic group with 1 to 6 carbon atoms, and the second group is substituted by one of the following substituted groups consisting of alkoxy, alkenyl, alkynyl, aryl, heteroaryl, aldehyde, amino, ester, ether, halide, hydroxyl, ketone, nitro, silyl, sulfo-oxo, sulfonyl, sulfone, sulfoxide, sulfate and thiol.

22. The ionic liquid according to claim 21, wherein the compound of the ionic liquid is of Formula 8: 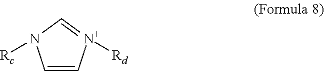
wherein R_{c} is —(Z¹)—O—(Z²)—OH, and Z¹ and Z² are individually alkylene with 1 to 6 carbon atoms, or substituted alkylene with 1 to 6 carbon atoms; R_{d} is —CₓH_{y}SO₃H, —CₓH_{y}PO₃H, —CₓH_{y}COOH, —CₓH_{y}SO₃⁻ or —CₓH_{y}COO⁻, and x ranges from 1 to 6 and y ranges from 2 to 12.

23. The ionic liquid according to claim 22, wherein the compound of the ionic liquid is of Formula 9: 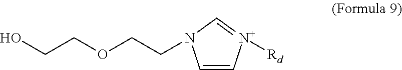
wherein, R_{d} is —CₓH_{y}SO₃⁻ or —CₓH_{y}COO⁻, x ranges from 1 to 6, and y ranges from 2 to 12.

24. The ionic liquid according to claim 22, wherein the compound of the ionic liquid is of Formula 10:

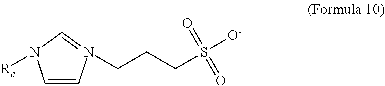

25. The ionic liquid according to claim 22, wherein the compound of the ionic liquid is of Formula 11: 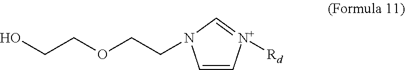
wherein, R_{d} is —CₓH_{y}SO₃H, —CₓH_{y}PO₃H or —CₓH_{y}COOH, x ranges from 1 to 6, and y ranges from 2 to 12.

26. The ionic liquid according to claim 25, wherein the ionic liquid further comprises an anion, and the anion comprises halogen ion, perchlorate, pseudohalogen ion, thiocyanate, cyanate, ClO₄⁻, BF₄⁻, PF₆⁻, AsF₆⁻, SbF₆⁻, NO₃⁻, NO₂⁻, SO₄²⁻, HSO₄⁻, PO₄³⁻, HPO₄²⁻, H₂PO₄⁻, CF₃CO₂⁻, CO₃²⁻, citrate, carboxylate with 1 to 6 carbon atoms and —CₓH_{y}COO, and x ranges from 1 to 6 and y ranges from 2 to 12.
